(19) United States
(12) Patent Application Publication
(10) Pub. No.: US 2009/0112187 A1
(43) Pub. Date: Apr. 30, 2009

(54) CATHETER RETENTION MECHANISM

(75) Inventors: Louis B. Goode, Cranberry Township, PA (US); Chun Kee Lui, Monroeville, PA (US); Christopher N. Magalich, Avonmore, PA (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook VascuIar Incorporated, Leechburg, PA (US)

(21) Appl. No.: 12/250,193

(22) Filed: Oct. 13, 2008

Related U.S. Application Data
(60) Provisional application No. 60/982,553, filed on Oct. 25, 2007.

(51) Int. Cl. A61M 25/16

(52) U.S. Cl. 604/533

(57) ABSTRACT

A retention mechanism for maintaining a position of a catheter on the connector tube of an implantable vascular access device. The retention mechanism includes an annular member dimensioned to be received over a length of the catheter when the catheter is positioned over the connector tube, and a housing for the annular member. The annular member is capable of elastic expansion for permitting passage of the member over a large diameter portion of the catheter, and elastic compression on the catheter for maintaining the position of the catheter on the connector tube. The housing has an inner passageway that includes a channel for receiving a radially outer portion of the annular member.

### RELATED APPLICATION

The present patent document claims the benefit of the filing date under 35 U.S.C. §119(e) of Provisional U.S. patent application Ser. No. 60/982,553, filed Oct. 25, 2007, which is hereby incorporated by reference.

### BACKGROUND

1. Technical Field

The present invention relates to an apparatus for securing a connection between two medical structures implanted in the body of a patient, and more particularly, to a retention mechanism for retaining an end of a catheter to the connector tube of an implantable vascular access port.

2. Background Information

Vascular access systems are utilized in the medical field for the transmission of fluids between a reservoir in an implanted vascular access port, and a body vessel in the patient. The vascular access port is surgically implanted by a physician into the subcutaneous tissue of a patient. Frequently, the port is implanted in the clavicular area of the chest, although it may also be implanted in other areas having sufficient underlying bony structure to provide support for the port. A catheter extends between the connector tube of the port and the body vessel, such as a blood vessel, for establishing fluid communication therebetween. The fluid generally comprises a liquid medicament that is injected into a reservoir of the vascular access port for use in treating a medical condition of the patient. Alternatively, the fluid can comprise a body fluid that is collected in the reservoir of the vascular access port for withdrawal and analysis. Typically, a vascular access port is implanted in a patient in situations in which it is expected that treatment of the patient with the medicament will continue for at least a period of several months. One of the most common uses of such ports is for cancer treatment, where a chemotherapeutic agent is injected into the reservoir, and transported therefrom through the catheter and vessel to a targeted body site.

A vascular access port typically includes a septum or similar penetrable closure through which the liquid medicament may be injected or otherwise transferred from the outside environment into the reservoir. When the port includes a septum, the septum generally comprises an elastomeric wall that covers all or a part of a surface of the port that is accessible to a needle. The septum is penetrated by the needle for injecting the medicament into the reservoir, or alternatively, for withdrawing blood or other body fluid from the reservoir.

A vascular access port is implanted into a pocket area of the subcutaneous tissue that has been deemed suitable for such use by the physician. The port is implanted in a manner such that the septum is readily accessible to the needle, so that the needle injection can be accomplished without undue complication and trauma to the patient. Once implanted, the port remains generally stationary in the pocket so that needle access to the port is not compromised. In many cases, the port is sutured or otherwise attached to available tissue in the body pocket to inhibit exceptional movement or shifting.

In a typical vascular access port, the catheter is connected to the port connector tube by first pushing the catheter over the connector tube, and thereafter pushing a rigid sleeve, typically fabricated from plastic or metal, over the catheter and the tube. This design has certain shortcomings. For example, during insertion of the sleeve over the catheter, the catheter is highly compressed between the minor diameter of the sleeve and the bead of the connector tube. The catheter then re-expands after the minor diameter has gone past the bead. The minor diameter creates a step which is employed to insure that the sleeve will not subsequently inadvertently withdraw from the connection. Since the only elasticity in this connection is in the compression of the catheter, the physician may have difficulty “feeling” that the connection has been properly effected. In addition, the high residual stresses created in the catheter render it susceptible to stress cracking over time. Further discussion of these deficiencies is provided in the description of the prior art designs illustrated in FIGS. 1 and 2 herein.

It would be desirable to provide a retention mechanism for a vascular access port that provides for secure attachment between the connector tube and the catheter, and that avoids the adverse occurrences of the prior art.

### SUMMARY

The present invention addresses the problems of the prior art. In one form thereof, the invention comprises a retention mechanism for maintaining a position of a catheter on the connector tube of an implantable vascular access device. The retention mechanism includes an annular member dimensioned to be received over a length of the catheter when the catheter is positioned over the connector tube, and a housing for the annular member. The annular member is capable of elastic expansion for permitting passage of the member over a large diameter portion of the catheter, and elastic compression on the catheter for maintaining the position of the catheter on the connector tube. The housing has an outer surface sized and configured for grasping by a physician and for insertion over the catheter, and has an inner passageway that includes a channel for receiving a radially outer portion of the annular member.

In another form thereof, the invention comprises an implantable assembly comprising a vascular access device, a catheter, and a retention mechanism for maintaining a position of the catheter over a connector tube of the vascular access device. The vascular access device includes a body portion having a fluid reservoir therein, wherein the connector tube is situated for conveyance of a fluid between the reservoir and a vessel of a patient. At least a portion of the connector tube extends outwardly from the body portion and includes a large diameter segment along a portion of its length. The catheter is sized such that an end of the catheter is receivable over the connector tube and the large diameter segment, and the other end is extendable into the vessel for conveyance of the fluid. The retention mechanism maintains the position of the catheter end over the connector tube. The retention mechanism includes an annular member sized to be received over the catheter end when the catheter end is positioned over the connector tube. The annular member is positioned intermediate the large diameter segment and the body portion along the length of the connector tube. The annular member is capable of elastic expansion for permitting passage of the annular member over the catheter and connector tube large diameter segment, and elastic compression on the catheter for maintaining the catheter position on the connector tube.

In still another form thereof, the invention comprises a retention apparatus for maintaining a position of a catheter on a connector tube of an implantable vascular access device. The implantable vascular access device includes a body portion, and the connector tube extends from the body portion for conveyance of a fluid through the catheter between the body portion and a vessel of a patient. The connector tube includes a larger diameter beaded portion along a length thereof having the catheter positioned thereover. The retention apparatus comprises a generally tubular member having an outer surface and an inner surface. The outer surface is sized and configured for grasping by a physician and for insertion over the catheter when the catheter is positioned over the connector tube. The inner surface defines a passageway extending axially therethrough and having a channel disposed along the passageway. The channel is dimensioned for receiving a radially expanded portion of the catheter positioned over the connector tube large diameter beaded portion.

### BRIEF DESCRIPTION OF THE DRAWINGS

FIG. 1 is a perspective view of a conventional vascular access port and catheter;

FIG. 2 is a cross-sectional view of the conventional vascular access port shown in FIG. 1, wherein a rigid sleeve is positioned over the catheter to maintain the position of the catheter over the port connector;

FIG. 3 is a cross-sectional view of a retention mechanism according to an embodiment of the present invention;

FIG. 4 is a cross-sectional view of a conventional vascular access port, wherein the retention mechanism of the present invention is positioned over the catheter;

FIG. 5 is a perspective view of an alternative embodiment of a retention mechanism according to the present invention;

FIG. 6 is a side view of the retention mechanism of FIG. 5; and

FIG. 7 is a cross-sectional view of the retention mechanism of FIG. 6.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

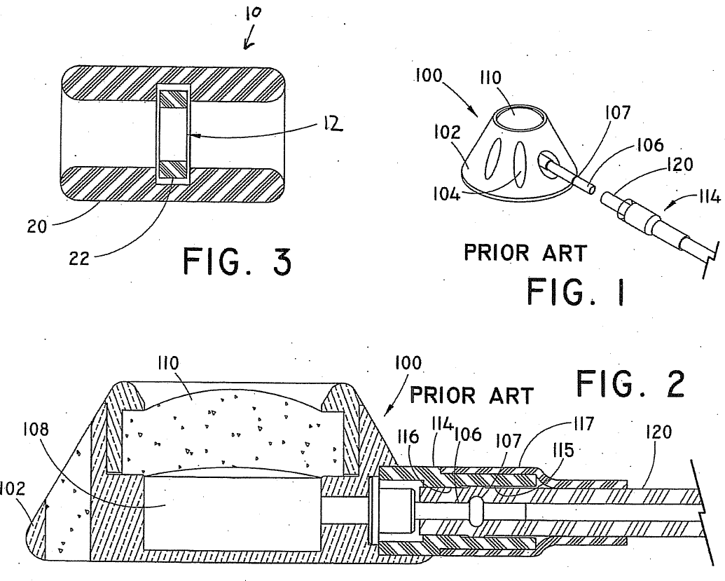

FIG. 1 is a perspective view of a conventional vascular access port 100 of the type that is well known in the art. Also illustrated in FIG. 1 is a conventional catheter 120 situated for placement over connector tube 106 of vascular access port 100 in well-known fashion. FIG. 2 illustrates a cross-sectional view of the conventional vascular access port 100 of FIG. 1, wherein the catheter 120 has been inserted over connector tube 106 to establish fluid communication between a body vessel and a fluid reservoir 108 disposed interiorly of vascular access port 100.

Conventional vascular access port 100 includes an outer main body portion 102 formed of a biocompatible material, such as titanium or polysulfone. Body portion 102 may be formed to include one or more suture holes 104 through which the physician may thread a suture to ligate the port to body tissue. A septum 110 is provided along an upper surface of vascular access port 100. Septum 110 generally comprises an elastomeric wall that is penetrable by a needle (not shown) to provide access to fluid reservoir 108. As shown in FIG. 2, a generally rigid tubular sleeve 114 is fitted over catheter 120 to inhibit disengagement of the catheter from the connector tube. In the version shown, sleeve 114 acts as a locking mechanism to prevent such disengagement. The locking mechanism may also include an outer silicone sleeve 117 that is press-fitted over sleeve 114. Vascular access devices are well known in the medical arts, and the vascular access port 100 illustrated and described herein is merely one example of such devices. The invention described herein may be utilized in connection with any such devices, provided that the device includes a connector tube similar to that described above.

In the prior art device shown, catheter 120 is connected to port connector tube 106 by first pushing the catheter over the exposed end of the connector tube. Sleeve 114 is thereafter inserted over the catheter and the tube, by pushing the sleeve in the direction of the arrow in FIG. 1. Sleeve 114 is typically formed of a rigid plastic or metal, and has a major inner diameter 115 and a minor inner diameter 116. Minor inner diameter 116 is provided at the leading edge of the sleeve, and, as shown in FIG. 2, defines a step which inhibits inadvertent withdrawal of sleeve 114 from the connection. As sleeve 114 passes over catheter 120, the portion of the catheter positioned directly over a larger-diameter beaded portion 107 of the connector tube becomes highly compressed when minor diameter 116 of rigid sleeve 114 passes over the bead. After minor diameter 116 clears this portion of the catheter, the catheter portion re-expands to its normal diameter. Since the only elasticity in this connection is in the compression of the catheter, the physician may have difficulty determining that the connection has been properly effected. In addition, the high residual stresses in the catheter, most notably in the highly compressed catheter portion described above, render it susceptible to stress cracking over time.

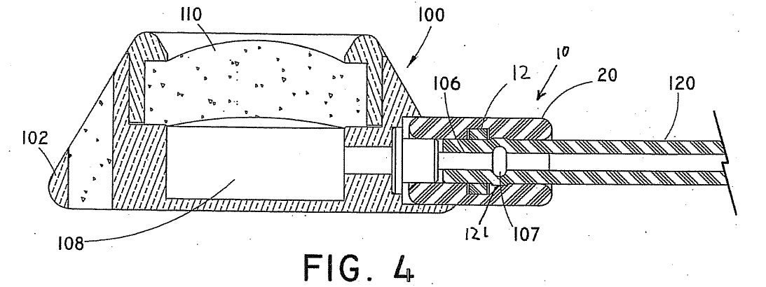

FIG. 3 illustrates a retention mechanism 10 according to an embodiment of the present invention. In the embodiment shown, retention mechanism 10 comprises an annular member 12 and a tubular housing 20. Preferably, housing 20 is provided with a channel 22 along the internal diameter of the housing, which channel is sized and shaped to receive an outer diameter of annular member 12. If desired, the outer surface of housing 20 may be shaped in any manner to enable it to be readily grasped and pushed by the physician during insertion of retention mechanism 10 over the catheter.

FIG. 4 illustrates use of the inventive retention mechanism to maintain the position of a catheter on a vascular access port. In this example, the port 100 and catheter 120 may be the same as shown in FIG. 2. One example of a suitable vascular access port 100 is the VITAL-PORT®, available from Cook Incorporated, of Bloomington, Ind. FIG. 4 differs from FIG. 2 in that catheter 120 is maintained in position over connector tube 106 by the inventive retention mechanism 10, rather than by the use of the rigid sleeve 114 as shown in FIG. 2.

Annular member 12 is formed from a composition, such as an elastomer, with sufficient elasticity to enable it to elastically expand as it is passed over catheter 120, and particularly over the larger diameter portion 121 of catheter 120 that is positioned directly over beaded portion 107 of the connector tube, during insertion of the retention mechanism as shown. Expansion of the annular member in this manner minimizes the stress induced upon the catheter, and particularly, upon larger diameter catheter portion 121. Once the annular member 12 of the retention mechanism has passed larger diameter catheter portion 121, the elasticity of the annular member causes it to compress onto the body of the catheter, thereby securing the catheter onto the connector tube. This elastic expansion and compression of the annular member give the physician the “feel” that the connection is being made, particularly as the mechanism “snaps” into a compressed condition as it traverses past the bead.

A particularly preferred elastomer for use in forming annular member 12 is silicone. Other materials having favorable elastic recovery properties, such as polyurethane, are acceptable alternatives. Annular member 12 can have virtually any cross-sectional geometry, the important feature being the ability to exert a retaining force on the catheter. In addition, annular member 12 may also be formed from other constructions capable of exerting a retaining force on the catheter. For example, annular member 12 may be fabricated from a helical spring, joined end-to-end to form a toroid. Alternatively, a split ring, or C-clip, which can perform the same function, may also be used. Typically, any structure that is capable of partially or completely enveloping the catheter, and is capable of expanding and thereafter recovering its dimension elastically, can be used as the annular member.

When fabricated from a material such as silicone or polyurethane, the annular member 12 can be molded. Alternatively, the annular member may be formed by slicing rings from silicone or polyurethane tubes. Structures such as toroids and C-clips can be fabricated from wires of titanium, stainless steel, nitinol, and the like.

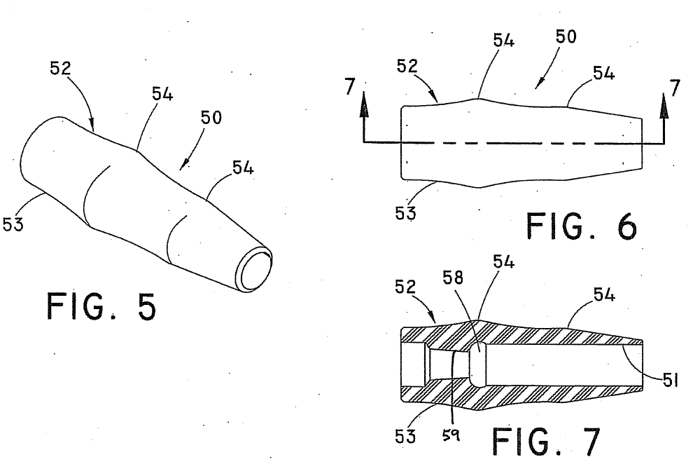

Housing 20 is structured to facilitate insertion of the annular member over the catheter 120 and connector tube 106. Utilizing housing 20 provides a convenient surface that can be readily grasped and inserted by the physician. If desired, the outer surface of housing 20 can be shaped in a manner to facilitate grasping (see, e.g., FIG. 5). Housing 20 can be fabricated from any material which possesses sufficient stiffness to retain the retention mechanism in the housing. Non-limiting examples include polysulfone, polyacetal, and silicone, each of which can be readily molded into a desired housing shape. Additionally, other material, such as titanium, can be readily machined into a desired shape.

As another variation, the features of an annular member and a housing may be combined in an integral structure. In this case, the retention mechanism may be fabricated from a single material, or alternatively, from multiple materials having differing properties. For example, an inner annular member portion, or layer, may be fabricated from a lower durometer elastomer, whereas an outer housing portion, or layer, may be fabricated from a higher durometer material, or a plastic.

An embodiment of an integral retention apparatus 50 is illustrated in FIGS. 5-7. Retention apparatus 50 can be formed from an elastic material, such as silicone or polyurethane, or from a combination of materials, such as described in the previous embodiment. When formed from the combination, the lower durometer inner material will have sufficient elasticity to receive the large diameter beaded portion of a catheter as described, whereas the higher durometer outer material will provide greater stiffness for maintaining the structural integrity of the device. Non-limiting examples of compositions for the inner material include materials having suitable elastic recovery properties, such as silicone, polyurethane, as well as other materials described above with reference to annular member 12. Non-limiting examples of compositions for the outer material include polysulfone, polyacetal, titanium, as well as other materials described above with reference to housing 20. Those skilled in the art are well aware of suitable procedures for forming a layered structure from these compositions (e.g., molding, extrusion, adhesion, etc).

In the preferred embodiment shown, retention apparatus 50 includes a generally tubular member 52 having an inner surface 51 and an outer surface 53. Outer surface 53 preferably has one or more ridges 54 to facilitate gripping by the physician. As shown in the sectional view of FIG. 7, the inner surface of tubular member 52 may be contoured, such as via the presence of optional channel 58. When retention apparatus 50 is positioned over the catheter, channel 58 can be positioned to receive a radially extended portion of catheter, such as catheter portion 121 shown in FIG. 4. Inner surface 51 may include a smaller diameter length 59 distal to channel 58. Smaller diameter length 59 is contoured to exert a retaining force on the catheter for maintaining the position of the catheter between the tubular member and the port connector tube. This arrangement provides a more gradual retention area when compared to the more discrete retention area of FIG. 4.

It is therefore intended that the foregoing detailed description be regarded as illustrative rather than limiting, and that it be understood that it is the following claims, including all equivalents, that are intended to define the spirit and scope of this invention.

1. A retention mechanism for maintaining a position of a catheter on a connector tube of an implantable vascular access device, the retention mechanism comprising:
an annular member dimensioned to be received over a length of said catheter when said catheter is positioned over the connector tube, said annular member being capable of elastic expansion for permitting passage of said annular member over the catheter, and elastic compression on said catheter for maintaining said catheter position on said connector tube; and
a housing for said annular member, said housing having an outer surface sized and configured for grasping by a physician and for insertion over said catheter, said housing having a passageway extending axially therethrough and having a channel disposed along said passageway, said channel dimensioned for receiving a radially outer portion of said annular member.

2. The retention mechanism of claim 1, wherein said connector tube includes a larger diameter beaded portion along a length thereof having said catheter positioned thereover, said annular member capable of elastic expansion over said beaded portion during passage of said annular member over said catheter.

3. The retention mechanism of claim 2, wherein said annular member comprises an elastomer.

4. The retention mechanism of claim 3, wherein said annular member comprises silicone or polyurethane.

5. The retention mechanism of claim 1, wherein said housing comprises polysulfone, polyacetal or silicone.

6. An implantable assembly, comprising:
a vascular access device, the vascular access device comprising a body portion having a fluid reservoir therein, and comprising a connector tube for conveyance of a fluid between the reservoir and a vessel of a patient, at least a portion of said connector tube extending outwardly from said body portion and including a large diameter segment along a portion of its length;
a catheter having first and second ends, said catheter sized such that said first end is receivable over said connector tube and said large diameter segment, and said second end is extendable into said vessel for conveyance of said fluid; and
a retention mechanism for maintaining a position of said catheter first end over said connector tube, said retention mechanism comprising an annular member sized to be received over said catheter first end when said catheter end is positioned over said connector tube, said annular member positioned intermediate said large diameter segment and said body portion along said length of said connector tube, said annular member being capable of elastic expansion for permitting passage of said annular member over the catheter and said connector tube large diameter segment, and elastic compression on said catheter for maintaining said catheter position on said connector tube.

7. The implantable assembly of claim 6, wherein said annular member comprises an elastomer.

8. The implantable assembly of claim 6, wherein said retention mechanism further comprises a housing for said annular member, said housing having an outer surface sized and configured for grasping by a physician and for insertion over said catheter when said catheter is positioned over said connector tube.

9. The implantable assembly of claim 8, wherein said housing has a passageway extending axially therethrough and has a channel disposed along said passageway, said channel dimensioned for receiving a radially outer portion of said annular member.

10. The implantable assembly of claim 8, wherein said annular member comprises silicone or polyurethane, and said housing comprises polysulfone, polyacetal or silicone.

11. The implantable assembly of claim 8, wherein said housing includes one or more ridges along an outer surface thereof.

12. A retention apparatus for maintaining a position of a catheter on a connector tube of an implantable vascular access device, the implantable vascular access device including a body portion, the connector tube extending from said body portion for conveyance of a fluid through said catheter between said body portion and a vessel of a patient, the connector tube including a larger diameter beaded portion along a length thereof having said catheter positioned thereover, the retention apparatus comprising:
a generally tubular member having an outer surface and an inner surface, said outer surface sized and configured for grasping by a physician and for insertion over said catheter when said catheter is positioned over said connector tube, said inner surface defining a passageway extending axially therethrough and having a channel disposed along said passageway, said channel dimensioned for receiving a radially expanded portion of said catheter positioned over said connector tube large diameter beaded portion.

13. The retention apparatus of claim 12, wherein at least a portion of said inner surface of said tubular member is capable of elastic expansion for permitting passage of said tubular member inner surface over said radially expanded catheter portion, and elastic compression on said catheter for maintaining said catheter position on said connector tube.

14. The retention apparatus of claim 12, wherein said tubular member is formed from silicone or polyurethane.

15. The retention apparatus of claim 12, wherein said outer surface includes one or more ridges for facilitating said grasping by the physician.

16. The retention apparatus of claim 12, wherein said inner surface defines a smaller diameter portion distal to said channel, said smaller diameter portion dimensioned for exerting a retaining force on a length of said catheter.

17. The retention apparatus of claim 12, wherein said outer surface comprises a material having a higher durometer than a durometer of a material of said inner surface.

18. The retention apparatus of claim 17, wherein said inner surface comprises an elastomer.

19. The retention apparatus of claim 18, wherein said outer surface comprises polysulfone, polyacetal, or titanium.

20. The retention apparatus of claim 19, wherein said outer surface includes one or more ridges for facilitating said grasping by the physician.
